(19) United States
(12) Patent Application Publication
(10) Pub. No.: US 2007/0038203 A1
(43) Pub. Date: Feb. 15, 2007

(54) Materials and methods for ablation of the endometrium

(76) Inventor: Jon T. McIntyre, Newton, MA (US)

Correspondence Address: FAY KAPLUN & MARCIN, LLP, 15O BROADWAY, SUITE 702, NEW YORK, NY, 10038, US

(21) Appl. No.: 11/178,569

(22) Filed: Jul. 11, 2005

(51) Int. Cl. A61B 18/18

(52) U.S. Cl. 606/14

(57) ABSTRACT

A system for ablating tissue lining a body cavity, comprises a source of diffusive medium including a plurality of scattering particles suspended therein and a medium introduction device coupled to the source of diffusive medium, a distal end of the medium introduction device being insertable into the body cavity for introduction of the diffusive medium thereinto in combination with a source of radiation energy which, when the system is in an operative configuration, remains outside the body and a wave guide coupled to the source of radiation energy to transmit the radiation energy from the source of radiation energy into the diffusive medium, a frequency of the radiation energy being matched with optical characteristics of the diffusive medium and scattering particles so that the radiation energy passes through the diffusive medium with out substantial absorption therein and is reflected from the scattering particles without substantial absorption therein. A method of ablating tissue comprises filling a body cavity with a diffusive medium including a plurality of scattering particles suspended therein and applying electromagnetic energy to the diffusive medium, a frequency of the electromagnetic radiation being matched to optical properties of the diffusive medium and the scattering particles so that the electromagnetic radiation is transmitted through the diffusive medium without substantial absorption therein and is reflected from the scattering particles without substantial absorption therein to disperse the electromagnetic radiation substantially uniformly over a surface of the body cavity to ablate a surface layer of tissue along the surface of the body cavity.

### BACKGROUND INFORMATION

Excessive or prolonged uterine bleeding during the menstrual cycle, also known as menorrhagia, affects a large number of women, mostly under the age of 45. Not all causes of excessive uterine bleeding are known, but some may include cervical or endometrial polyps, uterine fibroids, immune system diseases, pelvic inflammation and cervical or endometrial cancer.

Several treatment options are available for this condition, including drugs and surgical interventions. The surgical interventions are generally designed to remove a surface layer of the lining of the uterus (i.e., the endometrium). One treatment which has recently become available is endometrial ablation. Endometrial ablation involves the treatment of selected portions of tissue with laser light, heat, electric current, cryothermy, chemical solutions or other methods to necrose tissue which can then be removed surgically, by flushing, or in some other conventional manner. For example, a laser light source may be used to cause coagulation of the endometrial tissue in the spot where the laser light impinges on the tissue. The coagulation results in necrosis of the tissue, which may then be removed by, for example, a flushing step. However, the area which can be treated by a single laser light is very limited, requiring an repetitive, tedious procedure to treat the entire uterine cavity. Other systems, such as an RF roller ball which ablates the uterine lining by applying RF energy thereto, which locally treat the endometrium are also time consuming.

An alternative procedure is the global endometrial ablation, where all or most of the lining of the uterus is treated in one step. During this procedure, heat or another form of energy is applied to most or all of the endometrium, so that it is not necessary to individually treat small portions of the uterine wall. For example, hot water may be circulated through the uterus to ablate the endometrium or used to inflate a balloon inserted into the uterus. Circulating hot water is generally more effective in reachingall of the locations on the wall of the uterus since the water can follow all of the contours of the uterine cavity. However, it may take considerable time to sufficiently heat the circulating water. Using a balloon filled with water speeds the procedure. However, the surface of the balloon will not follow the inner contours of the uterus as accurately as free flowing water and may leave portions of the endometrium untreated.

### SUMMARY OF THE INVENTION

In one aspect, the present invention is directed to a system for ablating tissue lining a body cavity, comprises a source of diffusive medium including a plurality of scattering particles suspended therein and a medium introduction device coupled to the source of diffusive medium, a distal end of the medium introduction device being insertable into the body cavity for introduction of the diffusive medium thereinto in combination with a source of radiation energy which, when the system is in an operative configuration, remains outside the body and a wave guide coupled to the source of radiation energy to transmit the radiation energy from the source of radiation energy into the diffusive medium, a frequency of the radiation energy being matched with optical characteristics of the diffusive medium and scattering particles so that the radiation energy passes through the diffusive medium with out substantial absorption therein and is reflected from the scattering particles without substantial absorption therein.

The present invention is further directed to a method of ablating tissue comprising filling a body cavity with a diffusive medium including a plurality of scattering particles suspended therein and applying electromagnetic energy to the diffusive medium, a frequency of the electromagnetic radiation being matched to optical properties of the diffusive medium and the scattering particles so that the electromagnetic radiation is transmitted through the diffusive medium without substantial absorption therein and is reflected from the scattering particles without substantial absorption therein to disperse the electromagnetic radiation substantially uniformly over a surface of the body cavity to ablate a surface layer of tissue along the surface of the body cavity.

### BRIEF DESCRIPTION OF DRAWINGS

FIG. 1 is a diagram showing an embodiment of the endometrial ablation system according to the present invention, within a uterus of a patient.

### DETAILED DESCRIPTION

The present invention may be further understood with reference to the following description and the appended drawing, wherein like elements are referred to with the same reference numerals. The embodiments of the present invention are described in regard to methods and systems for ablating abnormal tissues within a patient's uterus. However, those skilled in the art will understand that the methods and devices according to this invention may be applied as well to ablation of the lining of any cavity in a patient's body.

Several medical procedures are available to treat menorrhagia, ranging from the administration of drugs to surgical interventions. When surgical procedures are recommended, one common approach is to remove the surface lining of the uterus, or endometrium, by ablating it.

In one exemplary embodiment according to the present invention, ablation of the endometrial lining is carried out by applying electromagnetic radiation, such as intense light energy, to the inner surface of the uterus. According to the embodiments, the cavity of the uterus is filled with a fluid, for example a liquid or a gel, which flows into the uterine cavity unconstrained by a balloon or by other containing devices. The fluid contains radiation scattering particles, which are mixed with the fluid to form a diffusive medium for the electromagnetic radiation.

Energy in the form of light is then introduced into the diffusive medium through an optical fiber or through another type of optical waveguide device connected to a light source. For example, the optical fiber may be inserted in the uterus via the cervical canal, and may have a distal end which terminates inside the uterus, within the diffusive medium. The optical fiber may, for example, be designed to fit within the working channel of a hysteroscopic instrument for insertion therethrough. Alternatively, a miniaturized source of electromagnetic radiation may be inserted into the uterus, and may be placed directly in contact with the diffusive medium.

Light is carried into the uterine cavity via, for example, the optic fiber enters the diffusive material and is scattered by the radiation scattering particles in all directions. The light exits the volume of the diffusive medium within the uterus in a substantially uniform manner, and impinges evenly on the inner lining of the uterus heating the tissue and coagulative necrosis of the lining, substantially uniformly along the entire surface of the endometrium. This type of procedure is effective in treating the abnormal uterine bleeding in one step. After the ablation procedure has been completed, the diffusive material is withdrawn from the uterus, and a flushing step may be carried out.

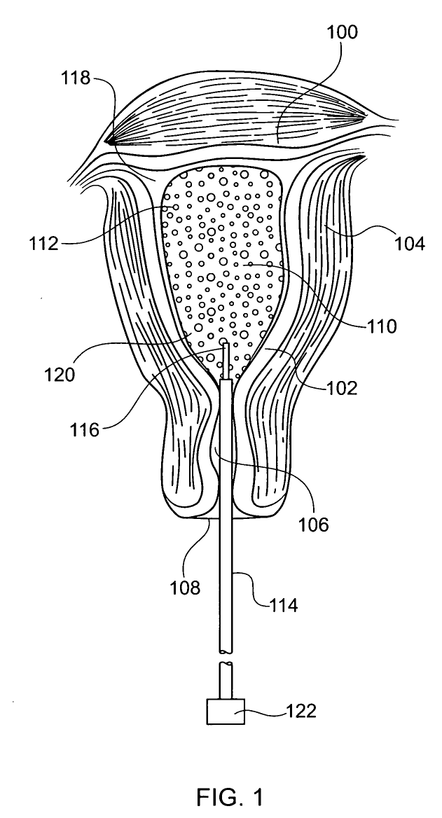

FIG. 1 shows an exemplary embodiment of the ablation system according to the present invention applied to a uterus 100. The uterus 100 is shown in cross section with the myometrium 104 and the endometrium 102 visible. As can be seen in FIG. 1, as the endometrium 102 is near the surface of the uterine lining, it can be effectively reached by radiation energy diffused from within the uterine cavity 118.

According to the exemplary embodiment, the diffusive medium 110 is introduced into the cavity 118 of the uterus 100 through the cervical opening 108 and the cervical canal 106 using, for example, a hysteroscopic instrument 114 or similar endoscopic device. The diffusive medium 110 comprises a fluid 120 such as water or a gel containing scattering particles 112 adapted to scatter electromagnetic energy introduced into the diffusive medium 110. For example, when high intensity light is used as the source of energy in the procedure, the scattering particles 112 may be formed of titania (e.g., titanium dioxide), silica or alumina. Additional materials may be used to form the scattering particles 112, to optimize to scattering properties for the type of energy that is introduced into the diffusive medium 110 as would be understood by those skilled in the art.

The diffusive medium 110 may be left stagnant in the uterus 100 during the duration of the treatment, so that flow handling devices are not required to move the fluid. Alternatively, the diffusive medium 110 may be circulated from a supply reservoir into and out of the uterine cavity 118 using conventional methods. The diffusive medium 110 according to the invention is designed to flow freely into the uterus 100, so that it follows every contour of the endometrium 102. Balloons or other constraining devices are not needed to contain the fluid 120 or prevent the fluid 120 from reaching all of the irregular regions of the anatomy of the uterus 100.

An elastomeric balloon formed, for example, of a material such as silicon, latex, etc., could also be used to house the diffusing medium, making it easier to completely withdraw the medium from the organ. An elastomeric material such as silicon or latex will also conform to the shape of the organ to assuring a more complete treatment and would absorb a low amount of light.

Once the diffusive medium 110 has been placed within the uterine cavity 118, a source of energy is engaged to begin transmitting and scattering through the medium, which is then absorbed by the tissue causing heating and then coagulation necrosis of the endometrium tissues to a depth of several millimeters. In one exemplary embodiment, an optic fiber 116 is used to convey light energy to the diffusive medium 110. For example, the optic fiber 116 may be introduced into the uterus 100 through the cervical opening 108 using the hysteroscopic instrument 114 with the optic fiber 116 extending beyond the distal opening of the hysteroscope 114. The optic fiber 116 is preferably placed in a location within uterine cavity 118 selected to provide substantially uniform irradiation of the diffusive medium 110 and, consequently, of the endometrium 102.

In one exemplary embodiment, suction may be applied to the distal end of the hysteroscopic instrument 114 to remove any air trapped at the interface between the optic fiber 116 and the surrounding diffusive medium 110. The vacuum may be provided in any conventional manner so that all gases may be removed from the interface between the optic fiber 116 and the diffusive medium 110. An optimal optical coupling may be promoted by applying the suction to keep the surface of the optic fiber 116 in contact with the fluid 120.

It will be apparent to those of skill in the art that alternatives to the optic fiber 116 may be used in embodiments of the present invention. For example, different types of wave guide devices may be used to convey light energy from a source into the diffusive medium 110. Wave guide devices may be used more generally to convey visible light or any other type of electromagnetic radiation from a source to the diffusive medium 110. A miniaturized source of high intensity light may be inserted into the uterus 100 and optically coupled to the diffusive medium 110. In another embodiment, either the source or a waveguide such as the optic fiber 116 may be inserted into the uterine cavity 118 independently of the hysteroscopic instrument 114.

As indicated above, a high power electromagnetic radiation source may be used to provide energy to the diffusive medium 110. For example, high intensity light having a wavelength of between about 900 nm and about 1064 nm may be passed through the optic fiber 116 and into the diffusive medium 110. A source 122 of the light energy may include a high power lightbulb, a laser, a diode, or other suitable source of radiation energy. When the light encounters the scattering particles 112 mixed in suspension in the fluid 120, it is deflected and eventually exits the diffusive medium 110. With the appropriate mixture of particles 112 and fluid 120, radiation is delivered to the endometrium 102 substantially uniformly over its entire surface as the diffusive medium 110 fills the entire volume of the uterine cavity 118.

To improve the performance of the system according to the present invention, the diffusive medium 110 may be optimized by selecting an appropriate fluid 120 and appropriate scattering particles 112. For example, the fluid 120 may be a gel or a liquid such as water, silicon gel, or deuterium oxide (D₂O). The fluid 120 is preferably selected to have an appropriate index of refraction for optimal coupling with the optical fiber 116, or with a similar light source. The fluid 120 is also preferably a fluid which is substantially transparent to light in the wavelength range used for the procedure to increase the efficiency of the ablation procedure. For example, D₂O is substantially transparent to light in the 900 nm to 1064 nm range. The scattering particles 112 are preferably selected for their ability to reflect light in the range of wavelengths used, without absorbing it. The density of the particles 112 in the fluid 120 is also preferably selected to provide a substantially uniform reflection of light in all directions onto the surface of the endometrium. Particles 112 also remain in suspension within fluid 120, and thus are selected to have a nearly neutral buoyancy.

As the diffusive medium 110 is in direct contact with the walls of the uterus 100, both the particles 112 and the fluid 120 are also non-toxic and biocompatible. For example, substances which are generally regarded as safe by the U.S. Food and Drug Administration (listed on the GRAS list) may be used to form the liquid 120 as well as the reflective particles 112.

After an ablation procedure has been completed, the diffusive medium 110 may be flushed out of the uterus 100, together with the ablated endometrial tissue. The endometrium 102 may then be inspected hysteroscopically to determine the effectiveness of the treatment. If necessary the treatment may be repeated without withdrawing the hysteroscope from the uterus 100. When further iterations of the procedure are no longer necessary (i.e., the desired degree of ablation has been achieved), the hysteroscope may be withdrawn to complete the procedure.

The present invention has been described with reference to specific exemplary embodiments. Those skilled in the art will understand that changes may be made in details, particularly in matters of shape, size, material and arrangement of parts. Accordingly, various modifications and changes may be made to the embodiments. Additional or fewer components may be used, depending on the condition that is being treated by the neurostimulation system. The specifications and drawings are, therefore, to be regarded in an illustrative rather than a restrictive sense.

What is claimed is:

1. A system for ablating tissue lining a body cavity, comprising:
a source of diffusive medium including a plurality of scattering particles suspended therein;
a medium introduction device coupled to the source of diffusive medium, a distal end of the medium introduction device being insertable into the body cavity for introduction of the diffusive medium thereinto;
a source of radiation energy which, when the system is in an operative configuration, remains outside the body; and
a wave guide coupled to the source of radiation energy to transmit the radiation energy from the source of radiation energy into the diffusive medium, a frequency of the radiation energy being matched with optical characteristics of the diffusive medium and scattering particles so that the radiation energy passes through the diffusive medium with out substantial absorption therein and is reflected from the scattering particles without substantial absorption therein.

2. The tissue ablation system according to claim 1, wherein the wave guide comprises an optic fiber.

3. The tissue ablation system according to claim 1, wherein the medium introduction device comprises an endoscopic instrument including a working lumen through which the waveguide may be inserted into the body cavity.

4. The tissue ablation system according to claim 3, further comprising a source of negative pressure coupleable to the endoscopic instrument to remove gases from an area adjacent to a distal end of the waveguide.

5. The tissue ablation system according to claim 1, wherein the diffusive medium comprises one of a liquid and a gel flowable at body temperature to fill anatomical irregularities of the body cavity.

6. The tissue ablation system according to claim 2, wherein the diffusive medium has a refractive index selected to optically couple with the optic fiber.

7. The tissue ablation system according to claim 1, wherein the diffusive medium comprises one of water, silicon gel, and deuterium oxide.

8. The tissue ablation system according to claim 1, wherein the scattering particles comprise at least one of silica, alumina and titania particles.

9. The tissue ablation system according to claim 1, wherein the medium introduction device includes an elastomeric balloon to enclose the diffusing medium.

10. The tissue ablation system according to claim 9, wherein the elastomeric balloon is formed of silicon.

11. The tissue ablation system according to claim 9, wherein the elastomeric balloon is formed of latex.

12. The tissue ablation system according to claim 1, wherein the source of the radiation energy emits electromagnetic radiation with a wavelength of between about 900 nm and about 1064 nm.

13. A method of ablating tissue comprising:
filling a body cavity with a diffusive medium, the diffusive medium including a plurality of scattering particles suspended therein;
applying electromagnetic energy to the diffusive medium, a frequency of the electromagnetic radiation being matched to optical properties of the diffusive medium and the scattering particles so that the electromagnetic radiation is transmitted through the diffusive medium without substantial absorption therein and is reflected from the scattering particles without substantial absorption therein to disperse the electromagnetic radiation substantially uniformly over a surface of the body cavity to ablate a surface layer of tissue along the surface of the body cavity.

14. The method according to claim 13, further comprising flushing the body cavity after the surface layer has been ablated.

15. The method according to claim 13, wherein a wavelength of the electromagnetic energy is between about 900 nm and about 1064 nm.

16. The method according to claim 13, wherein the body cavity is a uterus and wherein the surface layer is an endometrium.

17. The method according to claim 16, wherein the electromagnetic energy is applied to the diffusive medium by inserting into the uterus a wave guide coupled to a source of electromagnetic energy.

18. The method according to claim 17, wherein the wave guide includes a fiber optic cable.

19. The method according to claim 13, wherein the body cavity is filled with the diffusive medium through an endoscopic device inserted into the body cavity.

20. The method according to claim 19, wherein the electromagnetic energy is applied to the diffusive medium via a wave guide coupled to a source of electromagnetic energy and inserted into the uterus, wherein the endoscopic device includes a working through which the wave guide is inserted into the body cavity.

21. The method according to claim 17, further comprising applying a source of negative pressure an area adjacent to a distal end of the waveguide to remove gases therefrom.

22. The method according to claim 13, wherein the diffusive medium comprises one of a liquid and a gel flowable at body temperature to fill anatomical irregularities of the body cavity.

23. The method according to claim 17, wherein the diffusive medium has a refractive index selected to optically couple with the wave guide.

24. The method according to claim 13, wherein the diffusive medium comprises one of water, silicon gel, and deuterium oxide.

25. The method according to claim 13, wherein the scattering particles comprise one of silica, alumina and titania particles.

26. The method according to claim 25, wherein the scattering particles comprise titanium dioxide.

27. The method according to claim 13, wherein the electromagnetic energy has a wavelength of between about 900 nm and about 1064 nm.

28. The method according to claim 13, wherein the diffusing medium is enclosed within an elastomeric balloon within the body cavity.
